(19) United States
(12) Patent Application Publication
(10) Pub. No.: US 2026/0221236 A1
(43) Pub. Date: Jul. 30, 2026

(54) PREDICTION SYSTEM, PREDICTION PROGRAM, AND PREDICTION METHOD

(71) Applicant: ENEOS Corporation, Tokyo (JP)

(72) Inventor: Tasuku ONODERA, Tokyo (JP)

(21) Appl. No.: 19/136,143

(22) PCT Filed: Nov. 24, 2023
(86) PCT No.: PCT/JP2023/042257

§ 371 Date: Jun. 5, 2025

(30) Foreign Application Priority Data
Dec. 14, 2022 (JP) 2022-199612

(51) Int. Cl. G16C 20/30; G16C 10/00; G16C 20/70

(52) U.S. Cl. CPC G16C 20/30; G16C 10/00; G16C 20/70

(57) ABSTRACT

A prediction system according to the present invention includes an acquirer configured to acquire information regarding molecules included in a base oil that is liquid at normal temperature of 20° C. and normal pressure of 1.013×10⁵ Pa as molecules of interest, a single-molecule information acquirer configured to acquire a three-dimensional structure of the molecule of interest as single-molecule information, a liquid structure creator configured to create a liquid structure by defining a simulation cell to have a freely chosen parallelepiped shape and arranging a freely chosen number of units of the single-molecule information inside the simulation cell, an optimized structure acquirer configured to acquire a liquid optimized structure, a feature calculator configured to calculate a feature indicating an intermolecular space, and a predictor configured to predict a degree of density of an oil film formed of the molecules of interest based on the feature.

### TECHNICAL FIELD

The present invention relates to a prediction system, a prediction program, and a prediction method.

### BACKGROUND ART

In an industrial product provided with industrial machines, such as an internal combustion engine, hydraulic machine, compression machine, turbine, gear element, bearing, refrigerator, etc., in order to allow these machines to operate smoothly, various lubricants such as engine oil, hydraulic oil, compressor oil, turbine oil, gear oil, and refrigerating machine oil are used.

A lubricant mainly includes one or more base oils and one or more additives. As such a lubricant, for example, a lubricant composition containing an ester-based base oil and an additive for a lubricant, and the like are disclosed (for example, see Patent Document 1).

### RELATED ART DOCUMENTS

Patent Document 1: Japanese Unexamined Patent Application Publication No. 2022-158124

### SUMMARY OF THE INVENTION

### Problems to be Solved by the Invention

To allow a lubricant to exhibit desired lubricating performance, it is important to select a base oil that does not inhibit the effect of an additive or further enhances the effect of the additive. As a method of selecting a base oil, for example, there is a method of analyzing the structure of an oil film formed of the base oil by molecular simulation for each molecule constituting the base oil.

Specifically, as described in “Understanding the effect of the base oil on the physical adsorption process of organic additives using molecular dynamics” (M. Konishi and H. Washizu, Tribology International, 2020, vol. 149, article 105568), for example, there is a molecular dynamics simulation method, with which a diffusion behavior of an additive in a liquid phase of a base oil and an adsorption time of the additive to the surface of a metal plate are discussed, using an atomic/molecular scale model in which an additive is disposed in a liquid phase of a base oil and sandwiched between metal plates.

However, with this method, since it is necessary to perform complicated calculations, such as modeling and post-analysis, it takes a very long time to perform calculations, such as setting of analysis conditions, and it is not realistic to perform calculation for all molecules constituting a base oil. For example, in the case of analyzing the structure of an oil film formed of a base oil by molecular simulation for one type of base oil, it may take several days to several months until the calculation is completed. Therefore, a method for efficiently selecting a base oil that maximizes the effect of additives has been desired.

An objective of one aspect of the present invention is to shorten time required for selecting a base oil that brings out the effect of an additive.

### Means for Solving the Problem

One aspect of the present invention is a prediction system including: an acquirer configured to acquire molecules included in a base oil that is liquid at normal temperature of 20° C. and normal pressure of 1.013×10⁵ Pa as molecules of interest, a single-molecule information acquirer configured to acquire a three-dimensional structure of the molecule of interest as single-molecule information, a liquid structure creator configured to create a liquid structure by defining a simulation cell to have a freely chosen parallelepiped shape and arranging a freely chosen number of units of the single-molecule information inside the simulation cell, an optimized structure acquirer configured to acquire a liquid optimized structure optimized by relaxing the liquid structure, a feature calculator configured to calculate a feature indicating an intermolecular space by calculating an atomic distance between atoms of different molecules from the liquid optimized structure and calculating a radial distribution function representing an abundance ratio of one molecule to another molecule at each atomic distance, and a predictor configured to predict a degree of density of an oil film formed of the molecules of interest based on the feature.

Another aspect of the present invention is a prediction program for causing a computer to execute: an acquisition step of acquiring molecules included in a base oil that is liquid at normal temperature of 20° C. and normal pressure of 1.013×10⁵ Pa as molecules of interest, a single-molecule information acquisition step of acquiring a three-dimensional structure of molecules of interest as single-molecule information, a liquid structure creation step of creating a liquid structure by defining a simulation cell to have a freely chosen parallelepiped shape and arranging a freely chosen number of units of the single-molecule information inside the simulation cell, an optimized structure acquisition step of acquiring a liquid optimized structure optimized by relaxing the liquid structure, a feature calculation step of calculating a feature indicating an intermolecular space by calculating an atomic distance between atoms of different molecules from a liquid optimized structure and calculating a radial distribution function representing an abundance ratio of one molecule to another molecule at each atomic distance, and a prediction step of predicting a degree of density of an oil film formed of the molecules of interest based on the feature.

Another aspect of the present invention is a prediction method in which a computer is configured to execute: an acquisition step of acquiring molecules included in a base oil that is liquid at normal temperature of 20° C. and normal pressure of 1.013×10⁵ Pa as molecules of interest, a single-molecule information acquisition step of acquiring a three-dimensional structure of molecules of interest as single-molecule information, a liquid structure creation step of creating a liquid structure by defining a simulation cell to have a freely chosen parallelepiped shape and arranging a freely chosen number of units of the single-molecule information inside the simulation cell, an optimized structure acquisition step of acquiring a liquid optimized structure optimized by relaxing the liquid structure, a feature calculator of calculating a feature indicating an intermolecular space by calculating an atomic distance between atoms of different molecules from a liquid optimized structure and calculating a radial distribution function representing an abundance ratio of one molecule to another molecule at each atomic distance, and a prediction step of predicting a degree of density of an oil film formed of the molecules of interest based on the feature.

### Effects of the Invention

One aspect of the present invention shortens time required for selecting a base oil that brings out the effect of an additive.

### BRIEF DESCRIPTION OF THE DRAWINGS

FIG. 1 is an explanatory diagram illustrating prediction of a degree of density of an oil film from a molecule of interest.

FIG. 2 is an explanatory diagram illustrating a difference in adsorption states of an additive depending on a difference in a degree of density of an oil film.

FIG. 3 is a diagram illustrating a configuration of a prediction system according to an embodiment of the present invention.

FIG. 4 is a functional block diagram illustrating a configuration of a prediction device.

FIG. 5 is a diagram illustrating an example of a table in which structural formulae (SMILES) are described.

FIG. 6 is a diagram illustrating an example of creating a three-dimensional structure from a single-molecule structure of molecules of interest.

FIG. 7 is a diagram illustrating an example of creating a liquid structure from the three-dimensional structure of the molecule of interest.

FIG. 8 is a diagram illustrating an example of a radial distribution function.

FIG. 9 is a diagram illustrating an example of a distance between two molecules.

FIG. 10 is a diagram illustrating an example of a radial distribution function of a common normal alkane liquid.

FIG. 11 is a diagram illustrating an example of a radial distribution function focusing only on an atomic distance between molecules in FIG. 10.

FIG. 12 is a block diagram illustrating a hardware configuration of the prediction device.

FIG. 13 is a flowchart illustrating a prediction method according to the present embodiment.

### DESCRIPTION OF EMBODIMENTS

Hereinafter, embodiments of the present invention will be described in detail. In order to facilitate understanding of the description, the same reference numerals are given to the same components in the drawings, and redundant description will be omitted. In the present specification, “to” indicating a numerical range means that numerical values described before and after the “to” are included as a lower limit value and an upper limit value, unless otherwise specified.

### <Prediction System>

A prediction system according to an embodiment of the present invention will be described. The prediction system according to the present embodiment predicts a degree of density of an oil film formed of a base oil composed of molecules of interest.

In the present embodiment, molecules of interest are molecules making up a base oil that is liquid at normal temperature of 20° C. and normal pressure of 1.013×10⁵ Pa.

The base oil is an oil used as a base material for a lubricant, grease, or the like, and has a function of, for example, uniformly and stably dissolving an additive and feeding the additive to a place requiring it for lubrication. The base oil is mainly classified into mineral oils obtained by petroleum refining, synthetic oils obtained by chemical synthesis, mixed oils thereof, and the like. The lubricant or grease is a mixture containing one or more base oils and one or more additives. Examples of the lubricant include engine oils, hydraulic oils, compressor oils, turbine oils, gear oils, and refrigerator oils, which are used in industrial machines such as internal combustion engines, hydraulic machines, compression machines, turbines, gear elements, bearings, and refrigerators. Examples of the additives include plasticizers, stabilizers, oiliness agents, friction modifiers, anti-wear agents, antioxidants, ultraviolet absorbers, lubricants, mold release agents, antistatic agents, rust inhibitors, defoaming agents, viscosity index improvers, and the like.

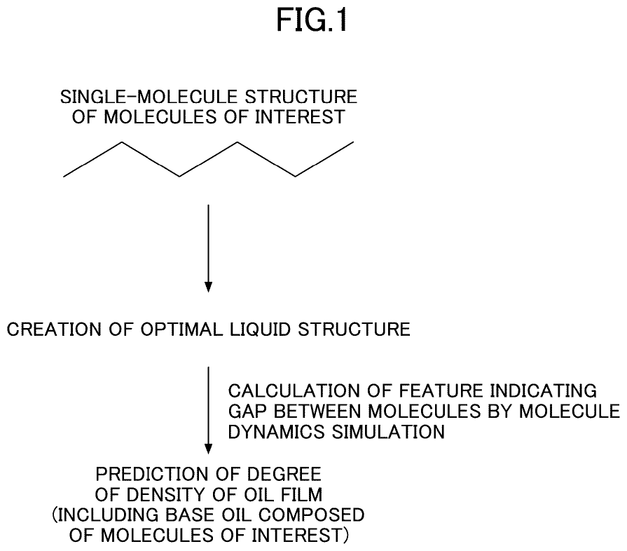

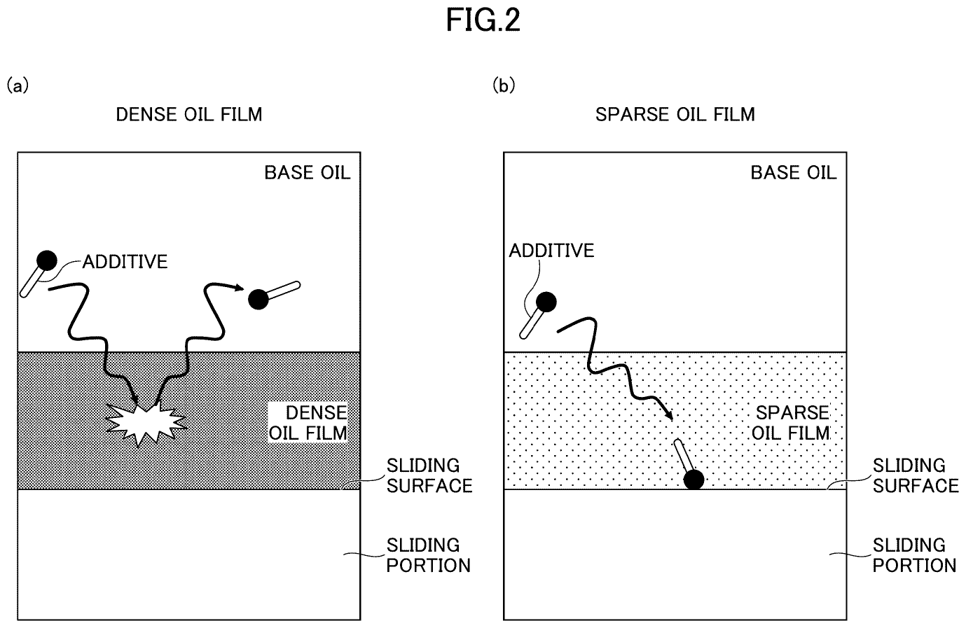

As illustrated in FIG. 1, the prediction system according to the present embodiment predicts a degree of density of an oil film containing molecules of interest by performing a molecular simulation such as a molecular dynamics method using an optimal liquid structure of a single-molecule structure of molecules of interest included in a base oil, and calculating a feature indicating an intermolecular space of the molecules of interest. As illustrated in FIG. 2, in a sliding portion of an industrial machine or the like, with respect to a sliding surface that rubs against a surface of another sliding component (hereinafter, simply referred to as a “sliding surface of a sliding portion”), as an oil film formed of a base oil composed of molecules of interest becomes denser, an additive contained in the base oil is less likely to pass through the inside of the oil film and is less likely to reach the sliding surface of the sliding portion; therefore, the effect of the additive tends to less likely be exhibited (see FIG. 2(a)). As the oil film becomes sparser, gaps are more likely to be generated in the oil film, and the additive is more likely to pass through the gaps in the oil film, and thus the additive migrates inside the oil film and easily reaches the sliding surface of the sliding portion (see FIG. 2(b)). Therefore, the effect of the additive, such as friction reduction, tends to be easily exhibited.

The prediction system according to the present embodiment predicts a degree of density of an oil film formed of molecules of interest by using, as a feature of the molecules of interest included in a base oil, a feature correlated with an atomic distance between atoms of different molecules (for example, a slope, a midpoint, or the like of an approximate expression of a radial distribution function, which will be described later), instead of an atomic distance in one molecule. The prediction system according to the present embodiment uses a prediction result of a degree of density of an oil film to select a base oil that brings out an effect of an additive, thereby shortening the time required for selecting a base oil.

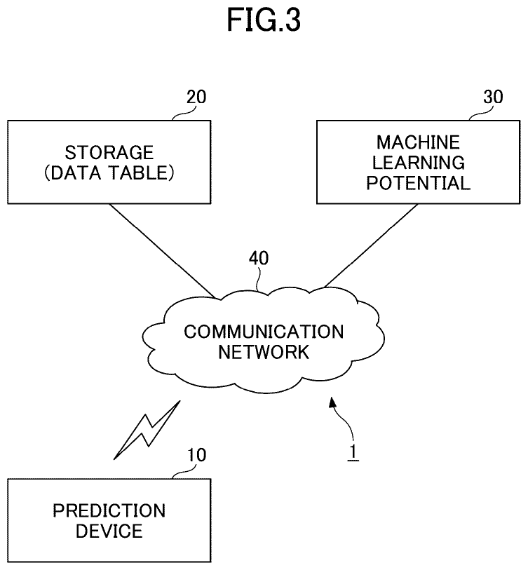

FIG. 3 is a diagram illustrating a configuration of the prediction system according to the present embodiment. As illustrated in FIG. 3, the prediction system 1 includes a prediction device 10, a storage 20, and a machine learning potential 30. In the prediction system 1, the prediction device 10, the storage 20, and the machine learning potential 30 are connected via a communication network 40, and input values to the prediction device 10, the storage 20, and the machine learning potential 30 and output values of the prediction device 10, the storage 20, and the machine learning potential 30 may be transmitted via the communication network 40. At least one of the storage 20 or the machine learning potential 30 may be stored on a cloud.

In the present embodiment, the prediction device 10, the storage 20, and the machine learning potential 30 are connected via the communication network 40, but may be connected in a wired or wireless manner. The prediction device 10, the storage 20, and the machine learning potential 30 may transmit and receive data via a communication network including a local area network (LAN), a wide area network (WAN), a personal area network (PAN), or the Internet. The prediction device 10 may be a single device such as a personal computer (PC) provided with components of the prediction device 10.

The prediction device 10 predicts a degree of density of an oil film formed of a base oil. Details of the prediction device 10 will be described later.

The storage 20 stores a data table including information regarding a lubricant and other information.

The information regarding a lubricant includes information regarding a type of a lubricant, a base oil and an additive included in the lubricant, and the like.

The type of a lubricant is engine oil, hydraulic oil, gear oil, refrigerator oil, or the like.

Examples of the information regarding a base oil and an additive include the types of a base oil and an additive, components and structural formulae constituting the base oil and the additive, and ratios of the base oil and the additive.

Examples of a base oil include mineral oils, synthetic oils, animal and plant oils, and mixed oils thereof.

Examples of an additive include plasticizers, stabilizers, oiliness agents, friction modifiers, anti-wear agents, metallic detergents, antioxidants, anti-friction and anti-wear agents, extreme pressure agents, ultraviolet absorbers, lubricants, mold release agents, ashless dispersants, oiliness improvers, antistatic agents, rust inhibitors, antifoaming agents, viscosity index improvers, metal deactivators, and solid lubricants.

Examples of components constituting a base oil include components constituting oils generally used as base oils, such as mineral oils, synthetic oils, animal and plant oils, and mixed oils thereof.

Examples of mineral oils include paraffin-based crude oils, naphthene-based crude oils, intermediate-based crude oils, aromatic-based crude oils, distillate oils, and refined oils. Examples of paraffin-based crude oils include isoparaffin and paraffin. Examples of naphthene-based crude oils include naphthene.

Examples of synthetic oils include poly-α-olefins, polyisobutylene (polybutene), monoesters, diesters, polyol esters, silicate esters, polyalkylene glycols, polyphenyl ethers, silicones, fluorine compounds, alkylbenzenes, and GTL base oils.

Examples of animal or vegetable oils include vegetable oils and fats such as castor oil, olive oil, cacao butter, sesame oil, rice bran oil, safflower oil, soybean oil, camellia oil, corn oil, rapeseed oil, palm oil, palm kernel oil, sunflower oil, cottonseed oil, and coconut oil, and animal oils and fats such as beef tallow, lard, milk fat, fish oil, and whale oil.

Examples of components constituting an additive include an ester compound of a monovalent or polyvalent aliphatic carboxylic acid and a monovalent or polyvalent aliphatic alcohol.

Examples of the monovalent aliphatic carboxylic acid used for the synthesis of an ester compound include saturated aliphatic carboxylic acids, such as methanoic acid, acetic acid, propionic acid, butyric acid, pentanoic acid, caproic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, icosanoic acid, henicosanoic acid, docosanoic acid, tricosanoic acid, tetracosanoic acid, pentacosanoic acid, hexacosanoic acid, heptacosanoic acid, octacosanoic acid, nonacosanoic acid, and triacontanoic acid, etc.

Examples of the polyvalent aliphatic carboxylic acid used for the synthesis of an ester compound include saturated aliphatic carboxylic acids, such as oxalic acid, malonic acid, succinic acid, glutaric acid, adipic acid, pimelic acid, suberic acid, azelaic acid, sebacic acid, undecanedioic acid, dodecanedioic acid, tridecanedioic acid, tetradecanedioic acid, heptadecanedioic acid, and hexadecanedioic acid.

Examples of the monovalent aliphatic alcohol used for the synthesis of an ester compound include methanol, ethanol, propanol, butanol, pentanol, hexanol, heptanol, octanol, nonanol, decanol, undecanol, dodecanol, tridecanol, tetradecanol, pentadecanol, and hexadecanol.

Examples of the polyvalent aliphatic alcohol used for the synthesis of an ester compound include ethylene glycol, propylene glycol, neopentyl glycol, glycerin, trimethylolethane, trimethylolpropane, pentaerythritol, and sorbitan.

The machine learning potential 30 is an interatomic potential using a machine learning method that outputs energy from information regarding a structure of an atom. Examples of the machine learning potential include a neural network potential (NNP), a Gaussian approximation potential (GAP), a spectral neighbor analysis potential (SNAP), and a moment tensor potential (MTP). The NNP is an interatomic potential obtained by learning a relationship between a structure (three-dimensional coordinates) of a molecule, a crystal, or the like and energy or force, which corresponds to an output, using deep learning. Among these, NNP is preferable as the machine learning potential in terms of high flexibility of neural network. As the NNP, Matlantis (registered trademark) may be used.

### (Prediction Device)

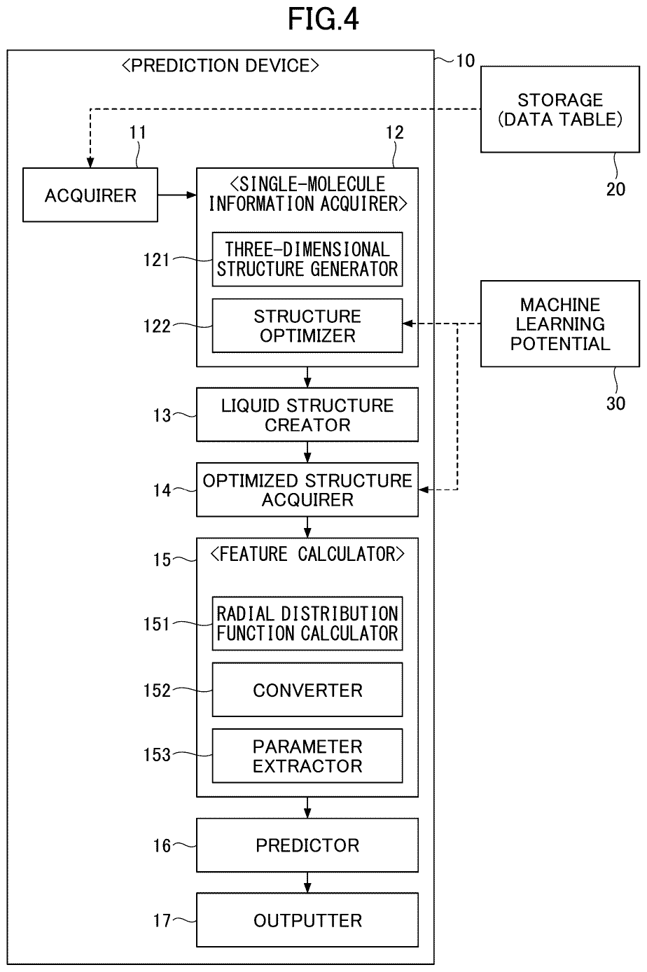

FIG. 4 is a functional block diagram illustrating a configuration of the prediction device 10. As illustrated in FIG. 4, the prediction device 10 includes an acquirer 11, a single-molecule information acquirer 12, a liquid structure creator 13, an optimized structure acquirer 14, a feature calculator 15, a predictor 16, and an outputter 17.

The acquirer 11 acquires information regarding molecules of interest that are molecules included in a base oil, and acquires information regarding an additive.

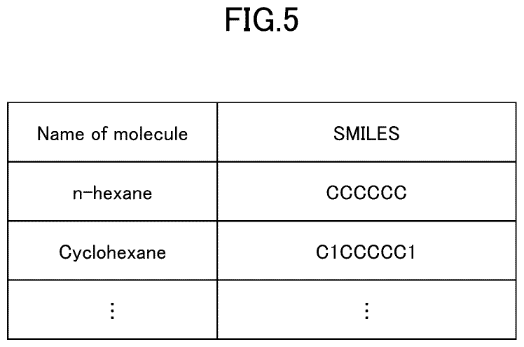

As information regarding the molecules of interest, name, structural formula, and the like of the molecules of interest may be used. As structural formula, SMILES or the like may be used. SMILES is a description of a molecular structure with a string of characters. An example of a table in which structural formulae (SMILES) are listed is illustrated in FIG. 5. As illustrated in FIG. 5, SMILES of molecules of interest are listed. The table containing the structural formulas of molecules of interest may be obtained from data in the format of CSV, spreadsheet software Excel, etc. The acquirer 11 may input a table in which SMILES of potential molecules of interest are written as illustrated in FIG. 5.

SMILES may be obtained from a chemical database, such as PubChem (a database of chemicals provided by NCBI in the United States).

As illustrated in FIG. 4, the single-molecule information acquirer 12 acquires a three-dimensional structure of the molecule of interest acquired by the acquirer 11 as single-molecule information.

The single-molecule information acquirer 12 includes a three-dimensional structure generator 121 and a structure optimizer 122.

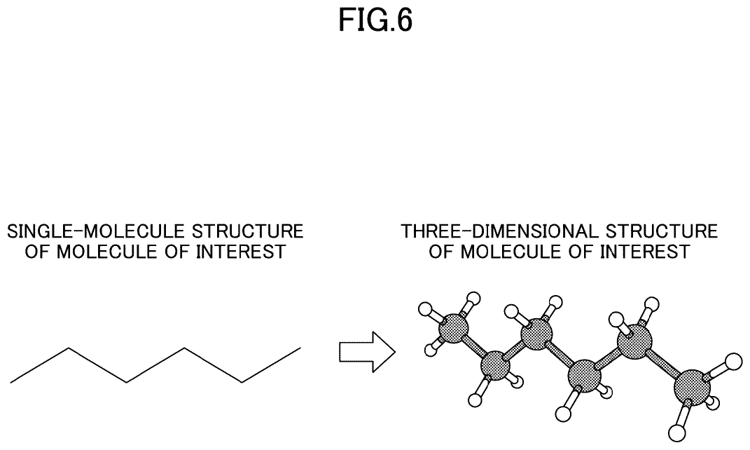

As illustrated in FIG. 6, the three-dimensional structure generator 121 generates a three-dimensional structure of a molecule of interest in which the hydrogens are added to the single-molecule structure of the molecule of interest by the three-dimensional structure generator 121.

The structure optimizer 122 acquires an optimized structure in which the three-dimensional structure is optimized, as the single-molecule information. The structure optimizer 122 arranges the atoms included in the molecule of interest at appropriate positions in consideration of the relationship between the coordinates and the energy of the atoms, and obtains a structure in which the three-dimensional structure is most stable in terms of energy.

The structure optimizer 122 creates an optimized structure, preferably using the machine learning potential 30. The three-dimensional structure generator 121 can increase the processing speed through using the machine learning potential 30, and thus can reduce the time required for creating an optimized structure.

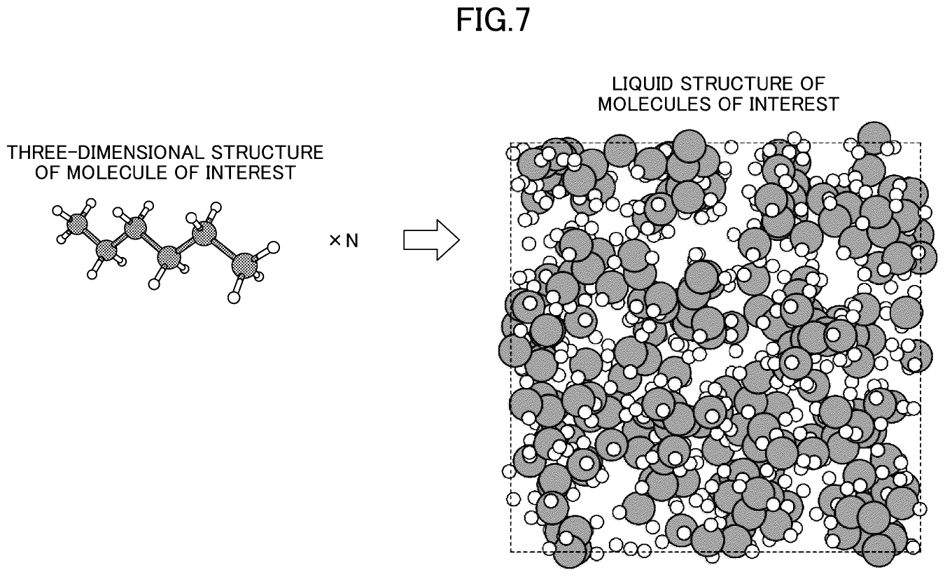

As illustrated in FIG. 4, the liquid structure creator 13 creates a liquid structure by arranging freely determined number (N) units of single-molecule information acquired by the single-molecule information acquirer 12 inside a simulation cell as illustrated in FIG. 7.

A simulation cell is defined to have the shape of any parallelepiped. The size of a simulation cell is freely set as appropriate according to the type of a lubricant or molecule of interest, or the like.

N is not particularly limited, and may be any number as appropriate, and may be, for example, on the order of several tens to several hundreds. If N is too small, the accuracy of the feature calculated by the feature calculator 15 is reduced. If N is too large, the calculation load becomes excessive and the calculation time is increased.

The liquid structure creator 13 arranges a freely determined number (N) of units of single-molecule information in a simulation cell at different positions so that the N units of single-molecule information do not overlap.

It is preferable that the liquid structure creator 13 define a simulation cell in such a manner that the actual structure of a lubricant is reproduced by making the density in the simulation cell substantially the same as the actual density of a base oil.

The liquid structure creator 13 may create a liquid structure by randomly arranging and rearranging a freely determined number (N) of units of single-molecule information in a simulation cell.

As illustrated in FIG. 4, the optimized structure acquirer 14 acquires a liquid optimized structure optimized by relaxing the liquid structure created by the liquid structure creator 13. Relaxation is a general structural optimization method, such as a steepest descent method or a conjugate gradient method, for acquiring a minimum value of energy in a multi-dimensional space. For example, relaxation means that a sum or scalar of force vectors, or a stress tensor or the main component of the stress tensor, or the like acting on the entire liquid structure is made to coincide with a predetermined pressure (external pressure), which is described later, under a certain threshold value. The liquid structure created by the liquid structure creator 13 does not have a correct density corresponding to an actual state because the single-molecule information is put in a simulation cell having an arbitrary size. With an incorrect density, the feature calculator 15 is highly likely to result in calculating a feature with low accuracy. The optimized structure acquirer 14 can create a liquid optimized structure adjusted to a liquid structure having a correct density by optimizing the liquid structure.

When optimizing the liquid structure, the optimized structure acquirer 14 preferably creates the liquid optimized structure under the following two conditions.
(1) When the optimized structure acquirer 14 optimizes the liquid structure, the volume of the simulation cell is variable. The optimized structure acquirer 14 may create a liquid structure at a predetermined pressure (external pressure) by applying an external force to the simulation cell.
(2) The optimized structure acquirer 14 creates a structure under the condition (1) as a structure at the absolute zero temperature. In order to take the influence of temperature into consideration, it is preferable to perform a molecular dynamics simulation.

The liquid optimized structure is created under the above-described two conditions. The simulation cell is thereby defined to reproduce a density of the actual base oil. In other words, the simulation cell is defined in such a manner that the liquid structure has a density when relaxed in consideration of a predetermined pressure and temperature.

Examples of the molecular dynamics simulation include structural optimization (a molecular mechanics method), a molecular dynamics method, and a Monte Carlo method.

To acquire a structure at the absolute zero temperature, the optimized structure acquirer 14 makes the volume of a simulation cell variable. The temperature may be set to any value as appropriate. The optimized structure acquirer 14 may create a liquid structure at a predetermined pressure (external pressure) by applying an external force to the simulation cell.

The temperature and pressure are preferably high, for example, when simulating a lubricant in a contact state. The high temperature is, for example, preferably 50° C. to 200° C., and more preferably about 60° C. The high pressure is preferably, for example, 50 MPa to 2000 MPa, and more preferably about 500 MPa, simulating real contact, such as contact between microprotrusions on one surface and microprotrusions on another surface.

The optimized structure acquirer 14 acquires a liquid stable structure of a molecule of interest, preferably using the machine learning potential 30. The optimized structure acquirer 14 can increase the calculation speed by using the machine learning potential 30, and thus can shorten the time required for acquiring a liquid stable structure.

The feature calculator 15 calculates a feature indicating an intermolecular space by calculating an atomic distance between atoms of different molecules from a liquid optimized structure and calculating a radial distribution function (RDF) representing an abundance ratio of one molecule to another molecule at each atomic distance. In other words, the feature calculator 15 may use a feature represented in a radial distribution function (for example, a slope, a midpoint, or the like of an approximate expression of a radial distribution function, which will be described later) represented based on an atomic distance between atoms of different molecules, instead of an atomic distance in one molecule.

An atomic distance between atoms of different molecules is a distance between two atoms included in two molecules respectively. Hereinafter, one molecule is referred to as “molecule A”, and another molecule is referred to as “molecule B”. An atomic distance between atoms of different molecules may be a distance between “atom a” included in molecule A and an atom existing at a position corresponding to atom a included in molecule B, or may be a distance between atom a included in molecule A and an atom existing at a position corresponding to an atom included in molecule B other than atom a.

The feature calculator 15 may include a radial distribution function calculator 151, a converter 152, and a parameter extractor 153.

The radial distribution function calculator 151 calculates an atomic distance between atoms of different molecules from a liquid optimized structure, and calculates a radial distribution function representing an abundance ratio of one molecule to another molecule at each atomic distance.

A radial distribution function is calculated by, for example, the following Expression (1):

$$\begin{matrix}[ Expression 1 ] & \\ {g}_{A - B} ( r ) = \frac{〈 {N}_{A - B} 〉}{4 π {r}^{2} \cdot Δ r \cdot {ρ}_{B}} & ( 1 )\end{matrix}$$

(wherein A is one molecule, B is another molecule different from molecule A, r is a radial distance as viewed from atom a of molecule A, g_{A-B}(r) is a probability of presence of atom b of molecule B existing at a radial distance r as viewed from atom a of molecule A, <N_{A-B}> is an ensemble average of the number of atoms b of molecule B existing in a section ranging from r−½Δr to r+½Δr as viewed from atom a of molecule A, Δr is a spherical section, and ρ_{B} is a number density of B.)

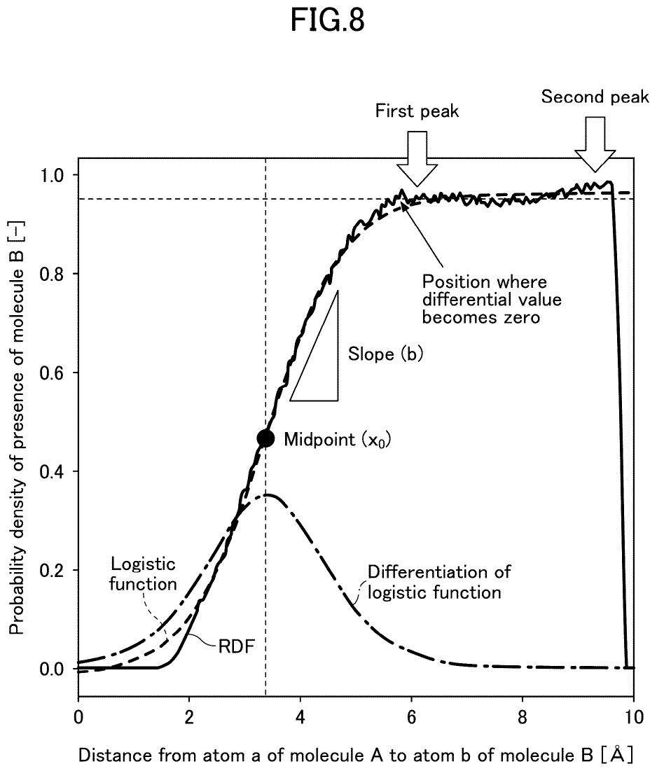

FIG. 8 shows an example of a radial distribution function illustrating a relationship between atom a of molecule A and atom b of molecule B. A radial distribution function is obtained in which a probability density of presence of molecule B rises from a constant state of 0 toward 1 as a distance from atom a of molecule A to atom b of molecule B increases as illustrated in FIG. 8, and the vertical axis value becomes substantially constant near 0.95 for a horizontal axis section illustrated in FIG. 8.

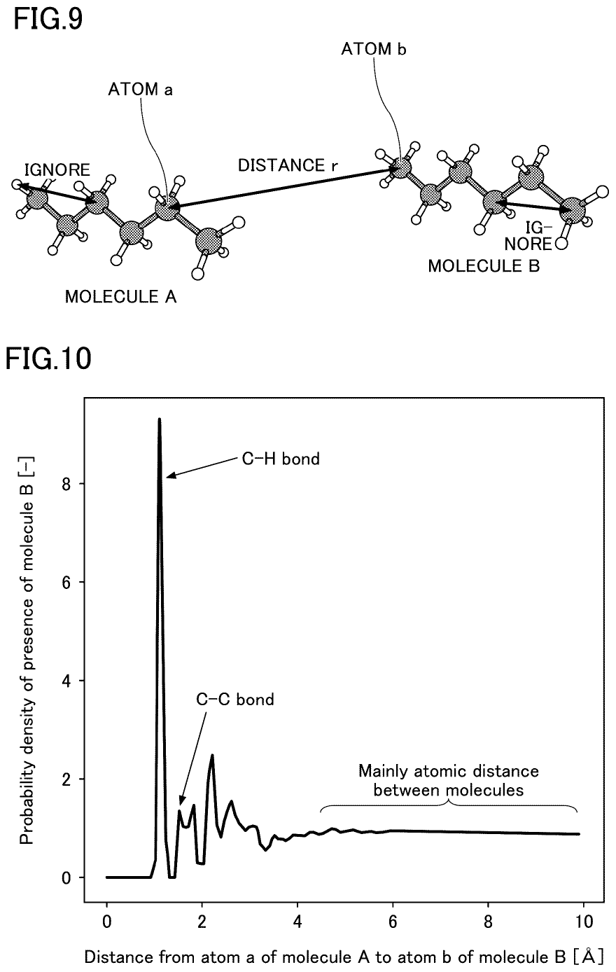

A radial distribution function indicates a probability that atom b of molecule B exists at a distance r from atom a of molecule A, as illustrated in FIG. 9. In other words, a radial distribution function represents a frequency of atom b of molecule B being positioned at a distance r from atom a of molecule A, and correlates with an intermolecular distance between atom a of molecule A and atom b of molecule B.

A radial distribution function may be plotted in multiple dimensions, instead of two dimensions.

The converter 152 converts the radial distribution function obtained by the radial distribution function calculator 151 into an approximate expression. The converter 152 may obtain an approximate expression of the radial distribution function by fitting the radial distribution function with a logistic function of the following Expression (2) or the like.

$$\begin{matrix}[ Expression 2 ] & \\ y = \frac{a}{1 + {e}^{- b ( x - {x}_{0} )}} + c & ( 2 )\end{matrix}$$

(wherein y is a probability density of presence of molecule B, a and c are coefficients, b corresponds to a slope of the approximate expression, x is a distance (Å) from an atom in molecule A to an atom in molecule B, and x₀ is the position of the midpoint of the slope of the approximate expression.)

The parameter extractor 153 extracts a parameter of the approximate expression of the radial distribution function converted by the converter 152, thereby calculating a feature indicating an intermolecular space. The parameter is preferably the midpoint or the slope of the approximate expression of the radial distribution function. In other words, in the case where the converter 152 obtains the approximate expression of the radial distribution function using the logistic function of the above Expression (2), the position of the midpoint of the approximate expression of the radial distribution function is used as the parameter x₀ and the slope of the approximate expression is used as the parameter b in the logistic function of Expression (2). Therefore, as illustrated in FIG. 8, the midpoint or the slope of the approximate expression of the radial distribution function can be used as a parameter of a feature indicating an intermolecular space, and the midpoint or the slope of the approximate expression of the radial distribution function is correlated with the atomic distance between atoms of different molecules and is correlated with the degree of density of the oil film.

A midpoint of an approximate expression corresponds to a distance between molecules. The larger a midpoint, the more molecules are separated from each other, which means that a relatively sparse oil film is formed, and the adsorption time until an additive is adsorbed on the surface is short.

A slope of an approximate expression corresponds to a distance between molecules. The smaller a slope, the more molecules are separated from each other, which means that a relatively sparse oil film is formed, and the adsorption time until an additive is adsorbed on the surface is short.

The smaller a midpoint of the approximate expression or the larger a slope, the closer molecules are, which means that a relatively dense oil film is formed, and the adsorption time until an additive is adsorbed on the surface is long.

Therefore, the adsorption time of an additive becomes shorter as a midpoint of an approximate expression becomes larger or a slope becomes smaller. If an additive can be adsorbed to the sliding surface of the sliding part of the industrial machine or the like in a shorter time, the effect of the additive can be exhibited, and the sliding surface of the sliding part can be protected from severe friction; therefore, effects such as low friction and low wear can be obtained. Therefore, it is preferable to adjust an oil film to have a sparse structure by increasing a midpoint of an approximate expression or decreasing a slope to increase an intermolecular space.

As illustrated in FIG. 8, the parameter extractor 153 may use, as a feature indicating an intermolecular space, a first peak (a local maximum value that appears first when viewed from a short distance to a long distance), a second peak (a local maximum value that appears second when viewed from a short distance to a long distance), or a value on the horizontal axis when a differential value of the radial distribution function asymptotically approaches substantially zero, of the radial distribution function.

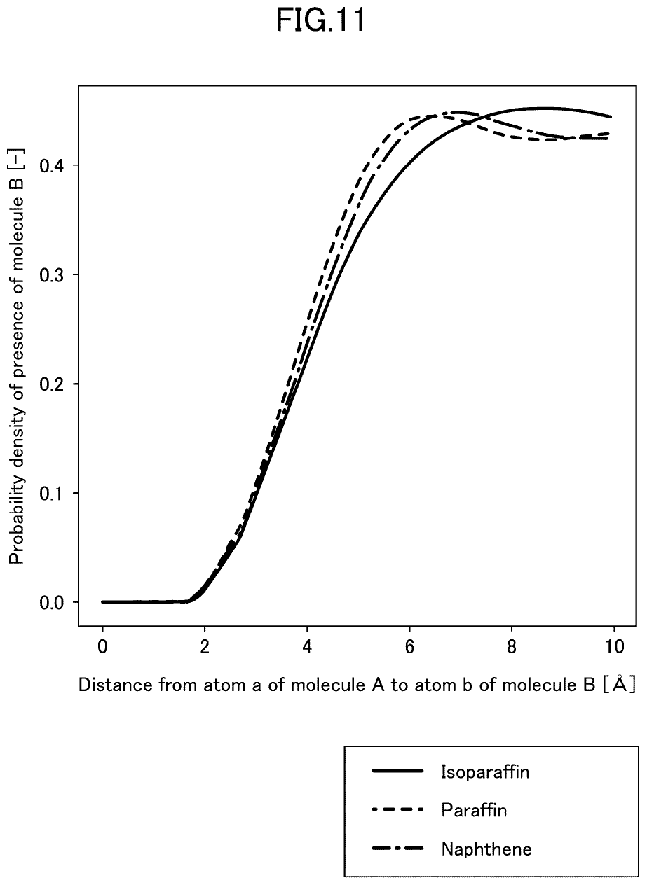

The feature calculator 15 uses a feature correlated with an atomic distance between different molecules (for example, a slope, a midpoint, or the like of an approximate expression of a radial distribution function), instead of an atomic distance in one molecule, as a feature indicating an intermolecular space between molecules of interest included in the base oil. The feature calculator 15 can specify an intermolecular space by obtaining a radial distribution function focusing only on an atomic distance between molecules, because completely different features appear depending on a type of molecules. For example, in the case of a radial distribution function of a common normal alkane liquid, as illustrated in FIG. 10, since many C—H bonds and C—C bonds are present in one molecule, sharp peaks derived from the C—H bonds and the C—C bonds are present in a region where the distance between atoms in one molecule is short. In a region where the distance between molecules is long, a peak or the like relating to an intermolecular pair is present, but such a peak is relatively weak as compared with the atomic distance in one molecule, and is hardly recognized. If the atomic distance in one molecule such as a C—H bond or a C—C bond is excluded, as illustrated in FIG. 11, a radial distribution function in which only the distance between atoms of different molecules appears is obtained. A radial distribution function that represents only the distance between atoms of different molecules, such as isoparaffin, paraffin, and naphthene, has a completely different shape depending on the type of molecule, and thus is effective in specifying a space between atoms of different molecules.

The feature calculator 15 may use various physical properties, such as viscosity, flash point, diffusion coefficient, and thermal conductivity of the base oil, in addition to the midpoint or the slope of the approximate expression of the radial distribution function, as a feature indicating an intermolecular space.

The predictor 16 predicts a degree of density of an oil film formed of molecules of interest based on a magnitude of a feature indicating an intermolecular space calculated by the feature calculator 15, such as the midpoint or the slope extracted from the radial distribution function, specifically, by measuring whether the feature of the molecules of interest is larger or smaller relative to a feature of a base oil currently used.

The outputter 17 outputs the prediction result of the density of the oil film formed of the base oil composed of the molecules of interest inside the simulation cell, which is predicted by the predictor 16, through display, transmission, or the like.

### (Hardware Configuration of Prediction Device 10)

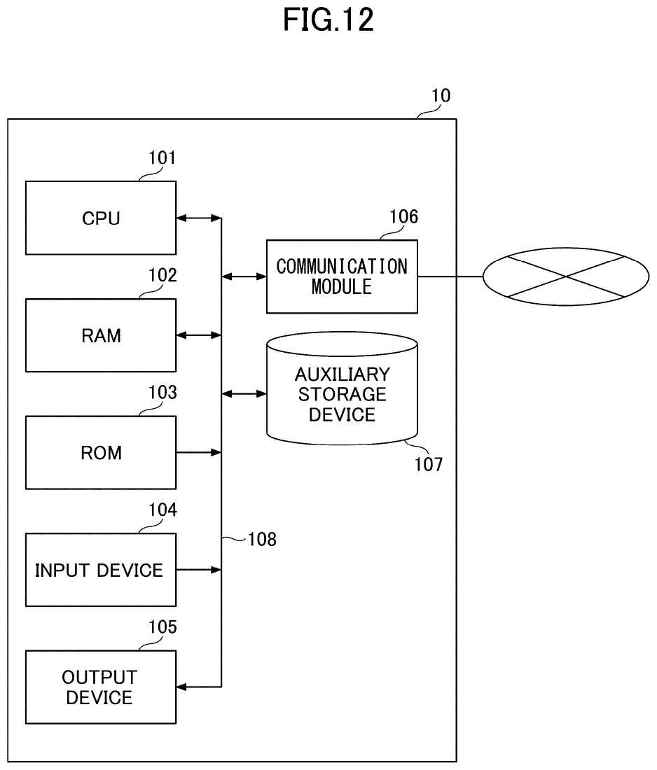

Next, an example of a hardware configuration of the prediction device 10 will be described. FIG. 12 is a block diagram illustrating a hardware configuration of the prediction device 10. As illustrated in FIG. 12, the prediction device 10 is configured by an information processing device (computer), and can be physically configured as a computer system including a central processing unit (CPU; a processor) 101 that is an arithmetic processing unit, a random access memory (RAM) 102 and a read only memory (ROM) 103 that are main storage devices, an input device 104 that is an input device, an output device 105, a communication module 106, an auxiliary storage device 107 such as a hard disk, and the like. These are connected to each other via a bus 108. The output device 105 and the auxiliary storage device 107 may be provided externally.

The CPU 101 controls the overall operation of the prediction device 10 and performs various types of information processing. The CPU 101 can predict a density of the oil film by, for example, performing a prediction method or executing a prediction program, which will be described later, stored in the ROM 103 or the auxiliary storage device 107.

The RAM 102 may include a non-volatile RAM that is used as a work area of the CPU 101 and stores main control parameters and information.

The ROM 103 stores a basic input/output program and the like. The prediction program may be stored in the ROM 103.

The input device 104 is an input device, such as a keyboard, a mouse, an operation button, a touch panel, or a display screen, receives information input by a user as an instruction signal, and outputs the instruction signal to the CPU 101.

The output device 105 is a display device such as a monitor display, a speaker, a printing device such as a printer, or the like. In the output device 105, for example, information such as the prediction result of the density of the oil film is displayed on a display device such as a monitor display, and a screen to be displayed is updated according to an input operation via the input device 104 or the communication module 106.

The communication module 106 is a data transmission/reception device such as a network card, and functions as a communication interface that takes in information from an external data recording server or the like and outputs analysis information to another electronic device.

The auxiliary storage device 107 is a storage device such as a solid state drive (SSD) and a hard disk drive (HDD), and stores, for example, various data, files, and the like necessary for the operation of the prediction device 10.

Each function of the prediction device 10 is realized by reading predetermined computer software (including a prediction program) from the main storage device such as the RAM 102 or the auxiliary storage device 107 and executing the computer software by the CPU 101 to read and write in the main storage device such as the RAM 102 or/and the auxiliary storage device 107 and the like and operate the input device 104, the output device 105, and the communication module 106.

Therefore, each part of the prediction device 10 illustrated in FIG. 4 is realized by cooperation of software and hardware by a processor executing predetermined computer software (including a prediction program) stored in advance in a computer including the prediction device 10.

A computer program implementing at least some of the functions of the parts of the prediction device 10 illustrated in FIG. 4 may be installed in a storage of one or more computers. The CPU 101 of one or more computers may read a computer program installed in the central processing unit into a main memory and execute the computer program to thereby exert the functions of the respective parts of the prediction device 10 illustrated in FIG. 4.

The prediction device 10 illustrated in FIG. 4 may be implemented by one or more CPUs 101. Herein, the CPU 101 may refer to one or more electronic circuits disposed on one chip, or may refer to one or more electronic circuits disposed on two or more chips or two or more devices. In the case where multiple electronic circuits are used, each electronic circuit may communicate by wired or wireless means.

The functions of the parts of the prediction device 10 illustrated in FIG. 4 may be executed by one computer or may be executed by a plurality of computers in a distributed manner. In the case where the functions of the respective parts of the prediction device 10 illustrated in FIG. 4 are executed in a distributed manner by a plurality of computers, the plurality of computers may transmit and receive data via a communication network including a local area network (LAN), a wide area network (WAN), a personal area network (PAN), or the Internet.

The prediction program can be stored in, for example, the main storage device or the auxiliary storage device 107 included in the computer. The prediction program may be stored in a computer connected to a communication line such as the Internet, and a part or all of the prediction program may be downloaded via the communication line. Furthermore, the prediction program may be configured to be provided or distributed via a communication line.

The prediction program may be recorded in (or installed on) the computer from a state where a part or all of the prediction program is stored in a portable storage medium such as an optical disk such as a CD-ROM and a DVD-ROM, a semiconductor memory such as a flash memory, or the like.

### <Prediction Method>

A prediction method according to the present embodiment will be described. The prediction method according to the present embodiment can be performed using the above-described prediction system 1. Therefore, the description of the matters already described will be partially omitted.

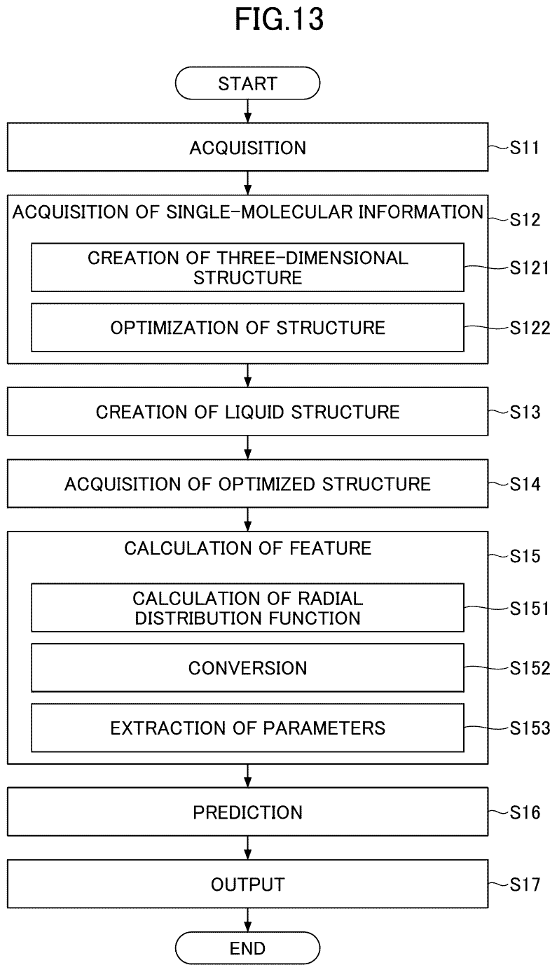

FIG. 13 is a flowchart illustrating the prediction method according to the present embodiment. As illustrated in FIG. 13, the prediction method according to the present embodiment is a prediction method for predicting a degree of density of an oil film formed of a base oil composed of molecules of interest.

In the prediction method according to the present embodiment, the acquirer 11 acquires information regarding molecules of interest, which are molecules included in the base oil, and acquires information regarding an additive (an acquisition step, step S11).

As described above, the name and the structural formula of the molecules of interest may be used as the information regarding the molecules of interest, and SMILES or the like may be used as the structural formula, as described above.

Next, the single-molecule information acquirer 12 acquires a three-dimensional structure of the molecules of interest acquired in the acquisition step (step S11) as single-molecule information (the single-molecule information acquisition step, step S12).

The single-molecule information acquisition step (step S12) may include a three-dimensional structure generation step (step S121) and a structure optimization step (step S122).

In the three-dimensional structure generation step (step S121), the three-dimensional structure generator 121 adds hydrogen to the single-molecule structure of the molecules of interest to generate a three-dimensional structure of the molecules of interest in which hydrogen is added to the single-molecule structure of the molecules of interest (see FIG. 6).

In the structure optimization step (step S122), the structure optimizer 122 acquires an optimized structure in which the three-dimensional structure is optimized as single-molecule information. In the structure optimization step (step S122), a structure in which the three-dimensional structure is most stable in terms of energy is obtained by the structure optimizer 122 arranging the atoms constituting the molecule of interest at appropriate positions in consideration of the relationship between the coordinates and the energy of the atoms.

In the structure optimization step (step S122), the structure optimizer 122 preferably acquires an optimized structure using the machine learning potential 30. In the structure optimization step (step S122), the time required for creating the optimized structure can be shortened by using the machine learning potential 30.

Next, the liquid structure creator 13 arranges N units of single-molecule information, which is a freely chosen number, acquired in the single-molecule information acquisition step (step S12) inside the simulation cell as illustrated in FIG. 7, thereby creating a liquid structure (the liquid structure creation step, step S13).

N is not particularly limited, and may be any number as appropriate, and may be, for example, on the order of several tens to several hundreds.

In the liquid structure creation step (step S13), the liquid structure creator 13 arranges a freely determined number (N) of units of single-molecule information in a simulation cell at different positions so that the N units of single-molecule information do not overlap.

In the liquid structure creation step (step S13), it is preferable that the liquid structure creator 13 define a simulation cell in such a manner that a density of the base oil is reproduced in the simulation cell.

In the liquid structure creation step (step S13), the liquid structure creator 13 may create a liquid structure by randomly arranging and rearranging a freely determined number (N) of units of single-molecule information in a simulation cell.

Next, the optimized structure acquirer 14 obtains a liquid optimized structure optimized by relaxing the liquid structure created in the liquid structure creation step (step S13) (the optimized structure acquisition step, step S14).

The liquid structure created in the liquid structure creation step (step S13) does not have a correct density corresponding to an actual state because the single-molecule information is put in a simulation cell having an arbitrary size. With an incorrect density, the feature calculation step (step S15) is highly likely to result in calculating a feature with low accuracy. The optimized structure acquisition step (step S14) can create a liquid optimized structure adjusted to a liquid structure having a correct density by optimizing the liquid structure.

In the optimized structure acquisition step (step S14), when optimizing the liquid structure, the optimized structure acquirer 14 may create the liquid optimized structure under the following two conditions.
(1) When the optimized structure acquirer 14 optimizes the liquid structure, the volume of the simulation cell is variable. The optimized structure acquirer 14 may create a liquid structure at a predetermined pressure (external pressure) by applying an external force to the simulation cell.
(2) The optimized structure acquirer 14 creates a structure under the condition (1) as a structure at the absolute zero temperature. In order to take the influence of temperature into consideration, it is preferable to perform a molecular dynamics simulation.

To acquire a structure at the absolute zero temperature, the optimized structure acquirer 14 makes the volume of a simulation cell variable. The temperature may be set to any value as appropriate. The optimized structure acquirer 14 may create a liquid structure at a predetermined pressure (external pressure) by applying an external force to the simulation cell.

The temperature and pressure are preferably high, for example, when simulating a lubricant in a contact state. The high temperature is, for example, preferably 50° C. to 200° C., and more preferably about 60° C. The high pressure is preferably, for example, 50 MPa to 2000 MPa, and more preferably about 500 MPa, simulating real contact, such as contact between microprotrusions on one surface and microprotrusions on another surface.

The optimized structure acquisition step (step S14) acquires a liquid stable structure of a molecule of interest, preferably using the machine learning potential 30. The optimized structure acquirer 14 can shorten the time required for acquiring a liquid stable structure by using the machine learning potential 30.

Next, the feature calculator 15 calculates a feature indicating an intermolecular space by calculating an atomic distance between atoms of different molecules from a liquid optimized structure and calculating a radial distribution function representing an abundance ratio of one molecule to another molecule at each atomic distance (the feature calculation step, step S15).

The feature calculation step (step S15) may include a radial distribution function calculation step (step S151), a conversion step (step S152), and a parameter extraction step (step S153).

In the radial distribution function calculation step (step S151), the radial distribution function calculator 151 calculates an atomic distance between atoms of different molecules from a liquid optimized structure, and calculates a radial distribution function representing an abundance ratio of one molecule to another molecule at each atomic distance.

A radial distribution function is calculated by, for example, the following Expression (1):

$$\begin{matrix}[ Expression 3 ] & \\ {g}_{A - B} ( r ) = \frac{〈 {N}_{A - B} 〉}{4 π {r}^{2} \cdot Δ r \cdot {ρ}_{B}} & ( 1 )\end{matrix}$$

(wherein A is one molecule, B is another molecule different from molecule A, r is a radial distance as viewed from atom a of molecule A, g_{A-B}(r) is a probability of presence of atom b of molecule B existing at a radial distance r as viewed from atom a of molecule A, <N_{A-B}> is an ensemble average of the number of atoms b of molecule B existing in a section ranging from r−½Δr to r+½Δr as viewed from atom a of molecule A, Δr is a spherical section, and ρ_{B} is a number density of B.)

In the conversion step (step S152), the converter 152 converts the radial distribution function obtained in the radial distribution function calculation step (step S151) into an approximate expression. In the conversion step (step S152), the converter 152 may obtain an approximate expression of the radial distribution function by fitting the radial distribution function with a logistic function of the following Expression (2) or the like.

$$\begin{matrix}[ Expression 4 ] & \\ y = \frac{a}{1 + {e}^{- b ( x - {x}_{0} )}} + c & ( 2 )\end{matrix}$$

(wherein y is a probability density of presence of molecule B, a and c are coefficients, b corresponds to a slope of the approximate expression, x is a distance (Å) from an atom in molecule A to an atom in molecule B, and x₀ is the position of the midpoint of the slope of the approximate expression.)

In the parameter extraction step (step S153), the parameter extractor 153 extracts a parameter of the approximate expression of the radial distribution function converted by the converter 152, thereby calculating a feature indicating an intermolecular space. The parameter is preferably the midpoint or the slope of the approximate expression of the radial distribution function (see FIG. 8). In other words, in the conversion step (step S152), in the case where the approximate expression of the radial distribution function is obtained using the logistic function of the above Expression (2), the position of the midpoint of the approximate expression of the radial distribution function is used as the parameter x₀ and the slope of the approximate expression is used as the parameter b in the logistic function of Expression (2). Therefore, as illustrated in FIG. 8, the midpoint or the slope of the approximate expression of the radial distribution function can be used as a parameter of a feature indicating an intermolecular space, and the midpoint or the slope of the approximate expression of the radial distribution function is correlated with the atomic distance between atoms of different molecules and is correlated with the degree of density of the oil film.

As illustrated in FIG. 8, in the parameter extraction step (step S153), as a feature indicating an intermolecular space, a first peak (a local maximum value that appears first when viewed from a short distance to a long distance), a second peak (a local maximum value that appears second when viewed from a short distance to a long distance), or a value on the horizontal axis when a differential value of the radial distribution function asymptotically approaches substantially zero, of the radial distribution function, may be used.

In the feature calculation step (step S15), the feature calculator 15 calculates a feature of the molecules of interest included in the base oil using a feature correlated with the atomic distances between different molecules (for example, a slope, a midpoint, and the like of the approximate expression of the radial distribution function). In the feature calculation step (step S15), the feature calculator 15 can specify an intermolecular space by obtaining a radial distribution function focusing only on an atomic distance between molecules, because completely different features appear depending on a type of molecules.

In the parameter extraction step (step S153), the feature calculator 15 may use various physical properties, such as viscosity, flash point, diffusion coefficient, and thermal conductivity of the base oil, in addition to the midpoint or the slope of the approximate expression of the radial distribution function, as the feature indicating an intermolecular space.

Next, the predictor 16 predicts a degree of density of the oil film formed of the molecules of interest based on the feature calculated in the feature calculation step (step S15) (the prediction step, step S16).

Next, the outputter 17 outputs, by display or the like, the prediction result of the density of the oil film formed of the base oil composed of the molecules of interest inside a simulation cell, which is predicted in the prediction step (step S16) (the outputting step, step S17).

As described above, the prediction system 1 includes the prediction device 10, and the prediction device 10 includes the acquirer 11, the single-molecule information acquirer 12, the liquid structure creator 13, the optimized structure acquirer 14, the feature calculator 15, and the predictor 16. The prediction system 1 acquires a three-dimensional structure of molecules of interest (see FIG. 6) as single-molecule information by the single-molecule information acquirer 12, creates a liquid structure in which the three-dimensional structure of the molecules of interest is arranged inside the simulation cell by the liquid structure creator 13 (see FIG. 7), and acquires a liquid optimized structure in which the liquid structure is optimized by the optimized structure acquirer 14. In the prediction system, the feature calculator 15 calculates an atomic distance between atoms of different molecules from a liquid optimized structure, and calculates a radial distribution function representing an abundance ratio of one molecule to another molecule at each atomic distance. In the prediction system 1, the predictor 16 predicts a degree of density of an oil film formed of the molecules of interest based on the calculated feature.

In the prediction system 1, a degree of density of an oil film can be predicted by calculating a feature from a radial distribution function obtained from a liquid optimized structure of molecules of interest, and thus a passage state of an additive in the oil film can be specified. In the case where an atomic distance between molecules is large in an oil film formed of a base oil composed of molecules of interest, gaps are likely to be generated. As an oil film has a sparser structure, an additive can migrate through the gaps in the oil film, and therefore, the additive can be quickly adsorbed to a sliding surface of a sliding portion of an industrial machine or the like with which the oil comes into contact, and the effect of the additive, such as the tribological characteristics, can be easily exhibited. Therefore, the prediction system 1 can shorten the time required to select a base oil that brings out an effect of an additive by predicting a degree of density of an oil film formed of molecules of interest, using a feature indicating an intermolecular space of the molecules of interest.

For example, in a case where a degree of density of an oil film formed of a base oil composed of molecules of interest is predicted using a diffusion/adsorption simulation, that has been generally used in the related art, it takes a long time (for example, on the order of several days to several months) to calculate a feature from a radial distribution function, even for one type of base oil, and thus it takes a longer time to predict a density of an oil film. The prediction system 1 can calculate a feature of one type of base oil in a short time of, for example, on the order of several tens of minutes to several hours, and thus can predict a degree of density of an oil film in a shorter time.

The prediction system 1 includes the predictor 16, and thus it is possible to reduce a burden and time required for predicting, from molecules of interest, a feature related to a density of an oil film formed of a base oil composed of molecules of interest. In production of a lubricant, in order to produce a lubricant that satisfies desired performance according to the type and use of the lubricant, in practice, a lubricant is produced by combining various base oils and additives in an experiment, and verification, etc. of a degree of density is performed. Since a type of a base oil that brings out an effect of an additive, a composition of a base oil and an additive, and the like are determined by performing these steps, a lot of labor is required, and the cost burden is large due to the preparation of various base oils and additives. The prediction system 1 can efficiently predict, from molecules of interest, a feature indicating an intermolecular space of the molecules of interest, while reducing the burden, and thus can reduce the burden when predicting a degree of density of an oil film formed of a base oil composed of the molecules of interest.

In the prediction system 1, the single-molecule information acquirer 12 may include the three-dimensional structure generator 121 and the structure optimizer 122. The three-dimensional structure generator 121 generates a three-dimensional structure in which hydrogen is added to molecules of interest included in a base oil, and the structure optimizer 122 optimizes the three-dimensional structure including the molecules of interest included in the base oil, and an optimized structure can be thereby acquired as single-molecule information. The three-dimensional structure generator 121 can create a three-dimensional structure appropriately representing a length, a size, an inclination, and the like of molecules included in a base oil, and the structure optimizer 122 can create a structure that appropriately reflects the relationship between the coordinates and energy of molecules of interest included in a base oil and is most stable in terms of energy. The prediction system 1 can more accurately provide a radial distribution function with the feature calculator 15 by using an optimized structure of molecules of interest obtained by the structure optimizer 122 as single-molecule information. Therefore, the prediction system 1 can more accurately calculate a feature of molecules of interest from a radial distribution function, and thus can more accurately predict a density of an oil film formed of a base oil composed of the molecules of interest. Therefore, the prediction system 1 can increase the accuracy in selection of a base oil that brings out an effect of an additive.

The prediction system 1 can use the machine learning potential 30 as the structure optimizer 122. Thus, the prediction system 1 can shorten the time required for the structure optimizer 122 to create an optimized structure of a three-dimensional structure of a molecule of interest. Therefore, the prediction system 1 can shorten the time required to predict a degree of density of an oil film, and thus can shorten the time required to select a base oil that brings out an effect of an additive.

In the prediction system 1, a simulation cell can be defined by the liquid structure creator 13 in such a manner that a density of a base oil is reproduced in the simulation cell. Thus, the prediction system 1 can bring a liquid structure of molecules of interest close to a state in which the molecules of interest are actually used as an oil such as a lubricant, and thus can bring a radial distribution function calculated by the feature calculator 15 close to the state of the lubricant that is actually used. Therefore, the prediction system 1 can calculate a feature from a radial distribution function so as to match a state of a base oil composed of molecules of interest that is actually used, and thus can predict a density of an oil film formed of the base oil with higher accuracy. Therefore, the prediction system 1 can more appropriately select a base oil that brings out an effect of an additive.

The prediction system 1 can use the machine learning potential 30 as the optimized structure acquirer 14. Accordingly, the prediction system 1 can shorten the time required for creating a liquid optimized structure in the optimized structure acquirer 14. Therefore, the prediction system 1 can shorten the time required to predict a degree of density of an oil film, and thus can shorten the time required to select a base oil that brings out an effect of an additive.

In the prediction system 1, the feature calculator 15 may include the radial distribution function calculator 151, the converter 152, and the parameter extractor 153. The feature calculator 15 can obtain a radial distribution function by the radial distribution function calculator 151, obtain an approximate expression by fitting the radial distribution function obtained by the converter 152, and use a parameter of the approximate expression of the radial distribution function for a feature based on the radial distribution function by the parameter extractor 153. Accordingly, the prediction system 1 can calculate a feature from a radial distribution function more easily and accurately, and thus can predict a density of an oil film more easily and accurately. Therefore, the prediction system 1 can select a base oil that brings out an effect of an additive with ease and increased accuracy.

The prediction system 1 can use a logistic function as an approximate expression of a radial distribution function in the parameter extractor 153, and a parameter may include a slope or a midpoint of the logistic function. Thus, the parameter extractor 153 can easily specify a slope or a midpoint of the approximate expression of the logistic function. Therefore, the prediction system 1 can specify a feature more easily and more accurately in the parameter extractor 153, and thus can predict a density of an oil film more easily and more accurately. Therefore, the prediction system 1 can more easily and appropriately select a base oil that brings out an effect of an additive.

As described above, the prediction system 1 can be used when selecting a base oil that brings out an effect of an additive, and thus can be suitably used for the production of a lubricant or the like. Since lubricants used in industrial machines and the like are used under severe conditions such as high pressure, high speed, high load, and high temperature, it is important to select a lubricant that can exhibit desired lubricating performance even under such environments. The prediction system 1 is used when selecting a base oil that brings out an effect of an additive, and thus it is possible to appropriately select a lubricant in accordance with the use of various lubricants used for industrial machines and the like.

Although the embodiments have been described above, the embodiments are presented as examples, and the present invention is not limited by the embodiments. The above embodiments can be implemented in various other forms, and various combinations, omissions, substitutions, changes, and the like can be made without departing from the gist of the invention. These embodiments and modifications thereof are included in the scope and gist of the invention, and are included in the invention described in the claims and the scope of equivalents thereof.

The embodiments of the present invention are as follows, for example.

<1> A prediction system, comprising:
an acquirer configured to acquire molecules included in a base oil that is liquid at normal temperature of 20° C. and normal pressure of 1.013×10⁵ Pa as molecules of interest;
a single-molecule information acquirer configured to acquire a three-dimensional structure of the molecule of interest as single-molecule information;
a liquid structure creator configured to create a liquid structure by defining a simulation cell to have a freely chosen parallelepiped shape and arranging a freely chosen number of units of the single-molecule information inside the simulation cell;
an optimized structure acquirer configured to acquire a liquid optimized structure optimized by relaxing the liquid structure;
a feature calculator configured to calculate a feature indicating an intermolecular space by calculating an atomic distance between atoms of different molecules from the liquid optimized structure and calculating a radial distribution function representing an abundance ratio of one molecule to another molecule at each atomic distance; and
a predictor configured to predict a degree of density of an oil film formed of the molecules of interest based on the feature.

<2> The prediction system according to <1>, wherein
the single-molecule information acquirer includes
   a three-dimensional structure generator configured to generate a three-dimensional structure of the molecules of interest by adding hydrogen to the molecules of interest; and
   a structure optimizer configured to acquire an optimized structure obtained by optimizing the three-dimensional structure as the single-molecule information.

<3> The prediction system according to <2>, wherein
the structure optimizer uses a machine learning potential.

<4> The prediction system according to any one of <1> to <3>, wherein
the liquid structure creator defines the simulation cell in such a manner that a density of the base oil is reproduced in the simulation cell.

<5> The prediction system according to any one of <1> to <3>, wherein
the optimized structure acquirer uses a machine learning potential.

<6> The prediction system according to any one of <1> to <5>, wherein
the feature calculator includes
   a radial distribution function calculator configured to calculate the radial distribution function;
   a converter configured to convert the radial distribution function into an approximate expression; and
   a parameter extractor configured to calculate the feature by extracting a parameter of the approximate expression.

<7> The prediction system according to <6>, wherein
the approximate expression is a logistic function, and
the parameter includes a slope or a midpoint of the logistic function.

<8> A prediction program for causing a computer to execute:
an acquisition step of acquiring molecules included in a base oil that is liquid at normal temperature of 20° C. and normal pressure of 1.013×10⁵ Pa as molecules of interest;
a single-molecule information acquisition step of acquiring a three-dimensional structure of molecules of interest as single-molecule information;
a liquid structure creation step of creating a liquid structure by defining a simulation cell to have a freely chosen parallelepiped shape and arranging a freely chosen number of units of the single-molecule information inside the simulation cell;
an optimized structure acquisition step of acquiring a liquid optimized structure optimized by relaxing the liquid structure;
a feature calculator calculates a feature indicating an intermolecular space by calculating an atomic distance between atoms of different molecules from a liquid optimized structure and calculating a radial distribution function representing an abundance ratio of one molecule to another molecule at each atomic distance; and
a prediction step of predicting a degree of density of an oil film formed of the molecules of interest based on the feature.

<9> A prediction method in which a computer is configured to execute:
an acquisition step of acquiring molecules included in a base oil that is liquid at normal temperature of 20° C. and normal pressure of 1.013×10⁵ Pa as molecules of interest;
a single-molecule information acquisition step of acquiring a three-dimensional structure of molecules of interest as single-molecule information;
a liquid structure creation step of creating a liquid structure by defining a simulation cell to have a freely chosen parallelepiped shape and arranging a freely chosen number of units of the single-molecule information inside the simulation cell;
an optimized structure acquisition step of acquiring a liquid optimized structure optimized by relaxing the liquid structure;
a feature calculation step of calculating a feature indicating an intermolecular space by calculating an atomic distance between atoms of different molecules from a liquid optimized structure and calculating a radial distribution function representing an abundance ratio of one molecule to another molecule at each atomic distance; and
a prediction step of predicting a degree of density of an oil film formed of the molecules of interest based on the feature.

This application claims priority based on Japanese Patent Application No. 2022-199612 filed with the Japan Patent Office on Dec. 14, 2022, the entire contents of which are incorporated herein by reference.

REFERENCE SIGNS LIST

| | |
|---|---|
| 1 | Prediction system |
| 10 | Prediction device |
| 11 | Acquirer |
| 12 | Single-molecule information acquirer |
| 13 | Liquid structure creator |
| 14 | Optimized structure acquirer |
| 15 | Feature calculator |
| 16 | Predictor |
| 17 | Outputter |
| 20 | Storage |
| 30 | Machine learning potential |
| 121 | Three-dimensional structure generator |
| 122 | Structure optimizer |
| 151 | Radial distribution function calculator |
| 152 | Converter |
| 153 | Parameter extractor |

1. A prediction system, comprising:
a storage storing a prediction program; and
a processor connected to the storage and configured to execute the prediction program, the prediction device including
an acquirer configured to acquire information regarding molecules included in a base oil that is liquid at normal temperature of 20° C. and normal pressure of 1.013×10⁵ Pa as molecules of interest;
a single-molecule information acquirer configured to acquire a three-dimensional structure of the molecule of interest as single-molecule information;
a liquid structure creator configured to create a liquid structure by defining a simulation cell to have a freely chosen parallelepiped shape and arranging a freely chosen number of units of the single-molecule information inside the simulation cell;
an optimized structure acquirer configured to acquire a liquid optimized structure optimized by relaxing the liquid structure;
a feature calculator configured to calculate a feature indicating an intermolecular space by calculating an atomic distance between atoms of different molecules from the liquid optimized structure and calculating a radial distribution function representing an abundance ratio of one molecule to another molecule at each atomic distance; and
a predictor configured to predict a degree of density of an oil film formed of the molecules of interest based on the feature.

2. The prediction system according to claim 1, wherein
the single-molecule information acquirer includes
a three-dimensional structure generator configured to generate a three-dimensional structure of the molecule of interest by adding one or more hydrogen atoms to the molecule of interest as needed; and
a structure optimizer configured to acquire an optimized structure obtained by optimizing the three-dimensional structure as the single-molecule information.

3. The prediction system according to claim 2, wherein
the structure optimizer uses a machine learning potential.

4. The prediction system according to claim 1, wherein
the liquid structure creator defines the simulation cell in such a manner that a density of the base oil is reproduced in the simulation cell.

5. The prediction system according to claim 1, wherein
the optimized structure acquirer uses a machine learning potential.

6. The prediction system according to claim 1, wherein
the feature calculator includes
a radial distribution function calculator configured to calculate the radial distribution function;
a converter configured to convert the radial distribution function into an approximate expression; and
a parameter extractor configured to calculate the feature by extracting a parameter of the approximate expression.

7. The prediction system according to claim 6, wherein
the approximate expression is a logistic function, and
the parameter includes a slope or a midpoint of the logistic function.

8. A non-transitory storage medium storing a prediction program for causing a computer to execute:
acquiring information regarding molecules included in a base oil that is liquid at normal temperature of 20° C. and normal pressure of 1.013×10⁵ Pa as molecules of interest;
acquiring a three-dimensional structure of the molecule of interest as single-molecule information;
creating a liquid structure by defining a simulation cell to have a freely chosen parallelepiped shape and arranging a freely chosen number of units of the single-molecule information inside the simulation cell;
acquiring a liquid optimized structure optimized by relaxing the liquid structure;
calculating a feature indicating an intermolecular space by calculating an atomic distance between atoms of different molecules from a liquid optimized structure and calculating a radial distribution function representing an abundance ratio of one molecule to another molecule at each atomic distance; and
predicting a degree of density of an oil film formed of the molecules of interest based on the feature.

9. A prediction method in which a computer is configured to execute:
acquiring information regarding molecules included in a base oil that is liquid at normal temperature of 20° C. and normal pressure of 1.013×10⁵ Pa as molecules of interest;
acquiring a three-dimensional structure of the molecule of interest as single-molecule information;
creating a liquid structure by defining a simulation cell to have a freely chosen parallelepiped shape and arranging a freely chosen number of units of the single-molecule information inside the simulation cell;
acquiring a liquid optimized structure optimized by relaxing the liquid structure;
calculating a feature indicating an intermolecular space by calculating an atomic distance between atoms of different molecules from a liquid optimized structure and calculating a radial distribution function representing an abundance ratio of one molecule to another molecule at each atomic distance; and
predicting a degree of density of an oil film formed of the molecules of interest based on the feature.
